(19) United States
(12) Patent Application Publication
(10) Pub. No.: US 2026/0115425 A1
(43) Pub. Date: Apr. 30, 2026

(54) SURFACE DISCONTINUITIES IN THE FORM OF ECHOGENIC MARKERS TO IMPROVE ECHOGENICITY ON ROUND MANDRELS

(71) Applicant: Boston Scientific Scimed, Inc., Maple Grove, MN (US)

(72) Inventors: Stephane Gareau Barnett, Toronto (CA); Owen Mitchell, Toronto (CA); Scott Zakaib, Toronto (CA); Kyle Kavaseri, Minneapolis, MN (US); Samarth Bhardwaj, Toronto (CA)

(21) Appl. No.: 19/368,721

(22) Filed: Oct. 24, 2025

Related U.S. Application Data
(60) Provisional application No. 63/712,330, filed on Oct. 25, 2024.

(51) Int. Cl. A61M 25/01; A61M 25/09; A61M 29/00

(52) U.S. Cl. CPC A61M 25/0108; A61M 25/09016; A61M 29/00; A61M 2025/09108; A61M 2025/09175; A61M 2210/125

(57) ABSTRACT

A transseptal guidewire with enhanced echogenicity and markers to facilitate precise positioning during transseptal puncture procedures. The guidewire comprises a flexible elongate body with a distal portion having echogenic markers formed by mechanical deformation of the guidewire surface, creating surface discontinuities that reflect ultrasound waves. These echogenic markers improve visibility under ultrasound imaging, allowing for accurate positioning of the guidewire tip at the target location. The guidewire also includes markers at the proximal end that provide visual or tactile feedback on the relative position of the guidewire with respect to a supporting member, such as a sheath or dilator. The proximal markers enable intuitive positioning and safe advancement of the guidewire during the procedure. By enhancing guidewire visibility and providing positioning feedback, improves the safety and efficiency of transseptal puncture procedures while maintaining compatibility with standard techniques and equipment.

### CROSS REFERENCE TO RELATED APPLICATIONS

This application claims priority to U.S. Provisional Patent Application No. 63/712,330 entitled “SURFACE DISCONTINUITIES IN THE FORM OF ECHOGENIC MARKERS TO IMPROVE ECHOGENICITY ON ROUND MANDRELS,” filed Oct. 25, 2024, which is hereby incorporated by reference in its entirety.

### TECHNICAL FIELD

The present disclosure relates to medical devices for puncturing tissue, such as the interatrial septum of the heart, using a guidewire with enhanced echogenicity. More specifically, the invention relates to guidewires with surface discontinuities or echogenic markers that improve visibility under ultrasound imaging, as well as methods for using such guidewires in transseptal puncture procedures. The echogenic markers on the guidewire enhance visibility during transseptal puncture procedures, allowing for precise positioning and navigation through the interatrial septum under ultrasound guidance.

### BACKGROUND

Transseptal puncture procedures are commonly performed to access the left atrium of the heart from the right atrium, allowing for a variety of diagnostic and therapeutic interventions. Traditionally, these procedures have relied on fluoroscopic imaging to guide the puncture device to the target location at the fossa ovalis and to visualize exchange guidewires in the left atrium once the device has crossed the septum. However, there is a growing trend towards using ultrasound imaging, such as intracardiac echocardiography (ICE) or transesophageal echocardiography (TEE), to reduce radiation exposure and improve soft tissue visualization.

One challenge with using ultrasound imaging in transseptal puncture procedures is that the guidewires and other ancillary devices can be difficult to visualize. This may be due to the smooth, rounded surfaces of conventional guidewires do not reflect ultrasound waves well, resulting in poor echogenicity. Poor visibility of the guidewire can make it difficult for the operator to determine the precise location of the body of the wire and to predict the trajectory of devices that are advanced over the guidewire. This can increase the risk of complications such as unintentionally puncturing adjacent structures.

To address this issue, some guidewires have been developed with enhanced echogenicity, such as a roughened surface, an echogenic coil or an echogenic coating. However, these guidewires still have limitations in terms of the degree of echogenicity that can be achieved and the manufacturing complexity.

There is a need for improved guidewires, such as RF wires for transseptal puncture, that have high echogenicity for clear visibility under ultrasound, while also being easy to manufacture. The echogenic features of the device should allow the operator to determine the location and trajectory of the device within the heart, without requiring fluoroscopy. The guidewire should also be compatible with standard transseptal puncture equipment and techniques.

### SUMMARY

In Example 1, a medical device for use in conjunction with an elongate guide member defining a lumen and having a proximal guide portion and a distal guide portion to cross a septum of a patient's heart, the device comprises a guidewire configured to be disposed within the lumen, the guidewire having an elongate body extending from a proximal end to a distal portion having a distal end, wherein the distal portion of the elongate body includes at least one echogenic feature, and wherein the at least one echogenic feature comprises a discontinuity configured to reflect ultrasonic waves so as to enhance visibility of the inner wire under ultrasound imaging.

In Example 2, the medical device of Example 1, wherein the at least one echogenic feature comprises a plurality of echogenic markers formed by mechanically deforming the surface of the distal portion of the elongate body to create a plurality of discontinuities.

In Example 3, the medical device of Example 2, wherein mechanically deforming the surface of the distal portion of the elongate body comprises creating a pattern of deformed material on the surface by forming indentations.

In Example 4, the medical device of any of Examples 2-3, wherein mechanically deforming the surface of the distal portion of the elongate body comprises at least one of laser etching, chemical etching, multi-way crimping, multi-way welding, material electro-spark deposition, grinding, grooving, knurling, threading, selective grit blasting, sink EDM, additive molding, or laser cutting.

In Example 5, the medical device of Example 3, wherein the indentations are configured to scatter ultrasound waves by presenting a non-uniform surface that reflects ultrasound waves in various directions.

In Example 6, the medical device of any of Examples 2-4, wherein the mechanical deforming creates at least two indentations in line, spaced 90° apart from each other around a circumference of the distal portion of the elongate body.

In Example 7, the medical device of Example 2, wherein the plurality of echogenic markers are evenly spaced along a length of the distal portion of the elongate body.

In Example 8, the medical device of Example 7, wherein the evenly spaced echogenic markers are configured to provide a visual reference for measuring anatomical features or an insertion depth of the guidewire under ultrasound imaging.

In Example 9, the medical device of Example 2, wherein the plurality of the echogenic markers are positioned to indicate different stiffness regions of the guidewire.

In Example 10, the medical device of Example 2, wherein a spacing between the plurality echogenic markers is selected to enable visualization of a relative motion of the guidewire under ultrasound imaging.

In Example 11, the medical device of Example 1, wherein the guidewire is configured to be advanced out of the distal guide portion of the elongate guide member.

In Example 12, the medical device of Example 1, wherein the elongate guide member comprises a dilator having a tapered distal end configured to facilitate advancement through the septum.

In Example 13, the medical device of Example 12, wherein the dilator further comprises at least one echogenic feature disposed on a surface of the dilator to enhance visibility of the dilator under ultrasound imaging.

In Example 14, the medical device of any of Examples 1 or 12-13, wherein the elongate guide member comprises a sheath having a distal end.

In Example 15, the medical device of Example 14, wherein the sheath further comprises at least one echogenic feature disposed on a surface of the sheath to enhance visibility of the sheath under ultrasound imaging.

In Example 16, a medical device for use in conjunction with an elongate guide member defining a lumen and having a proximal guide portion and a distal guide portion, the device comprises a guidewire configured to be disposed within the lumen, the guidewire having an elongate body extending from a proximal end to a distal portion having a distal end, wherein the distal portion of the elongate body includes at least one echogenic feature, and wherein the at least one echogenic feature comprises a discontinuity configured to reflect ultrasonic waves so as to enhance visibility of the inner wire under ultrasound imaging.

In Example 17, the medical device of Example 16, wherein the at least one echogenic feature comprises a plurality of echogenic markers formed by mechanically deforming the surface of the distal portion of the elongate body to create a plurality of discontinuities.

In Example 18, the medical device of Example 17, wherein mechanically deforming the surface of the distal portion of the elongate body comprises creating a pattern of deformed material on the surface by forming indentations.

In Example 19, the medical device of Example 17, wherein mechanically deforming the surface of the distal portion of the elongate body comprises at least one of laser etching, chemical etching, multi-way crimping, multi-way welding, material electro-spark deposition, grinding, grooving, knurling, threading, selective grit blasting, sink EDM, additive molding, or laser cutting.

In Example 20, the medical device of Example 18, wherein the indentations are configured to scatter ultrasound waves by presenting a non-uniform surface that reflects ultrasound waves in various directions.

In Example 21, the medical device of Example 18, wherein the mechanical deforming creates at least two indentations, spaced 90° apart from each other around a circumference of the distal portion of the elongate body.

In Example 22, the medical device of Example 17, wherein the plurality of echogenic markers are evenly spaced along a length of the distal portion of the elongate body.

In Example 23, the medical device of Example 22, wherein the evenly spaced echogenic markers are configured to provide a visual reference for measuring anatomical features or an insertion depth of the guidewire under ultrasound imaging.

In Example 24, the medical device of Example 17, wherein the plurality of the echogenic markers are positioned to indicate different stiffness regions of the guidewire.

In Example 25, the medical device of Example 17, wherein a spacing between the plurality of echogenic markers is selected to enable visualization of a relative motion of the guidewire under ultrasound imaging.

In Example 26, the medical device of Example 16, wherein the guidewire is configured to be advanced out of the distal guide portion of the elongate guide member.

In Example 27, the medical device of Example 16, wherein the elongate guide member comprises a dilator having a tapered distal end configured to facilitate advancement through the septum.

In Example 28, the medical device of Example 27, wherein the dilator further comprises at least one echogenic feature disposed on a surface of the dilator to enhance visibility of the dilator under ultrasound imaging.

In Example 29, the medical device of Example 16, wherein the elongate guide member comprises a sheath having a distal end.

In Example 30, the medical device of Example 29, wherein the sheath further comprises at least one echogenic feature disposed on a surface of the sheath to enhance visibility of the sheath under ultrasound imaging.

In Example 31, an ultrasound imaging system comprises: an ultrasound transducer configured to emit and receive ultrasound signals; and a medical device for use in conjunction with an elongate guide member defining a lumen and having a proximal guide portion and a distal guide portion to cross a septum of a patient's heart, the device comprising: a guidewire configured to be disposed within the lumen, the guidewire having an elongate body extending from a proximal end to a distal portion having a distal end, wherein the distal portion of the elongate body includes at least one echogenic feature, and wherein the at least one echogenic feature comprises a discontinuity configured to reflect ultrasonic waves so as to enhance visibility of the inner wire under ultrasound imaging.

In Example 32, the ultrasound imaging system of Example 31, wherein the at least one echogenic feature comprises a plurality of echogenic markers formed by mechanically deforming a surface of the distal portion of the elongate body to create the plurality of discontinuities.

In Example 33, the ultrasound imaging system of Example 32, wherein mechanically deforming the surface of the distal portion of the elongate body comprises at least one of laser etching, chemical etching, multi-way crimping, multi-way welding, material electro-spark deposition, grinding, grooving, knurling, threading, selective grit blasting, sink EDM, additive molding, or laser cutting.

In Example 34, the ultrasound imaging system of Example 32, wherein mechanically deforming the surface of the distal portion of the elongate body comprises creating a pattern of deformed material on the surface by forming indentations.

In Example 35, a method of manufacture a medical device with enhanced visibility under ultrasound imaging, the method comprising: providing an elongate guide member defining a lumen and having a proximal guide portion and a distal guide portion; providing a guidewire having an elongate body extending from a proximal end to a distal portion having a distal end; and forming at least one echogenic feature on the distal portion of the elongate body by mechanically deforming a surface of the distal portion to create a plurality of discontinuities that increase ultrasound wave reflection or beam scattering, thereby enhancing visibility of the guidewire under ultrasound imaging.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of an exemplary medical ultra sound system including a medical device with a guidewire for performing transseptal puncture procedure, in accordance with some embodiments disclosed herein.

FIG. 2 is a perspective view of the medical device, in accordance with some embodiments disclosed herein.

FIG. 3A a close-up top view of a portion of the guidewire of the medical device, illustrating the echogenic markers as surface discontinuities, in accordance with some embodiments disclosed herein.

FIG. 3B is a close-up side view of the portion of the guidewire shown in FIG. 3A, depicting the depth and spacing of the echogenic markers, in accordance with some embodiments disclosed herein.

FIG. 4 is a schematic representation of the interaction between an ultrasound imaging system and the guidewire with echogenic markers, illustrating the enhanced visibility achieved through the surface discontinuities of the echogenic marker, in accordance with some embodiments disclosed herein.

FIG. 5 is a side view of the guidewire, illustrating the echogenic markers and the varying stiffness regions along the length of the guidewire, in accordance with some embodiments disclosed herein.

FIG. 6 is a perspective view of the medical device in use within the heart, demonstrating the application of the echogenic markers on the guidewire as a measurement guide and positioning aid during a transseptal puncture procedure, in accordance with some embodiments disclosed herein.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The field of interventional cardiology has seen significant advancements in recent years, particularly in the development of minimally invasive techniques for accessing and treating various cardiac structures. One such procedure that has gained widespread acceptance is transseptal puncture, which involves creating a small hole in the interatrial septum to access the left atrium of the heart from the right atrium. This technique enables a wide range of diagnostic and therapeutic interventions, such as left atrial appendage closure, atrial fibrillation ablation, and mitral valve repair.

However, performing a transseptal puncture can be challenging, as it requires precise navigation and positioning of the puncture device within the heart. Traditionally, transseptal punctures have been performed under fluoroscopic guidance, which exposes the patient and the operator to ionizing radiation. Moreover, fluoroscopy provides only limited soft tissue visualization, making it difficult to accurately target the desired puncture site and avoid damaging adjacent structures.

To address these limitations, there has been a growing interest in using ultrasound imaging to guide transseptal puncture procedures. Ultrasound offers several advantages over fluoroscopy, including improved soft tissue visualization, real-time imaging, and the absence of ionizing radiation. However, conventional transseptal puncture devices can be difficult to visualize under ultrasound due to their small size and the acoustic properties of the materials used in their construction.

The present disclosure seeks to overcome these challenges by providing an improved transseptal puncture device and system that enhances the visibility of the guidewire under ultrasound imaging while also facilitating precise positioning and puncture of the target tissue.

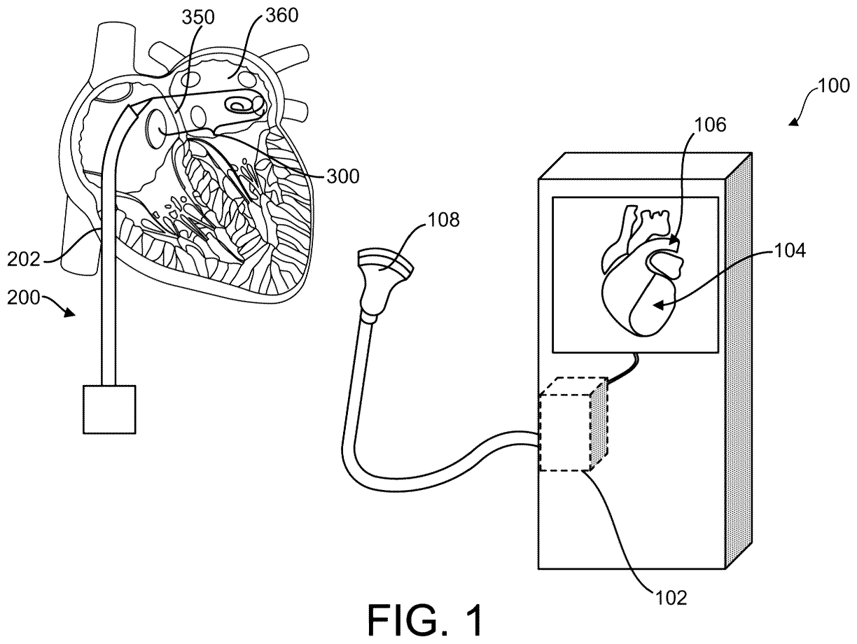

FIG. 1 illustrates an exemplary medical ultrasound system 100 for intracardiac procedures such as transseptal puncture. The system 100 includes an ultrasound data processor 102, an imaging system 106, and an ultrasound probe 108. For transseptal puncture procedures, the ultrasound probe 108 may be made for intracardiac or transesophageal use, allowing for high-resolution imaging from within the body. These specialized probes can be positioned in the left or right atrium, as with intracardiac echocardiography (ICE), or in the esophagus, as with transesophageal echocardiography (TEE). The ultrasound probe 108 is coupled to the ultrasound data processor 102 and is responsible for transmitting high frequency sound waves into the patient's body and receiving the reflected sound waves. The ultrasound data processor 102 analyzes the received waves and generates a visual representation 104 of the patient's anatomy, which is displayed on the imaging system 106.

The visual representation 104 provides a real-time, two-dimensional representation of the patient's heart, enabling the medical team to visualize the interior of the heart and the position of the medical device 200 within it. As shown in FIG. 1, the medical device 200 is positioned inside the right atrium of the heart, with the guidewire 300 extending through the interatrial septum 350 and into the left atrium 360. The visual representation 104 may take various forms including two-dimensional or three-dimensional images, real-time video feeds, or different models depending on specific capabilities of the system and the requirements of the procedure.

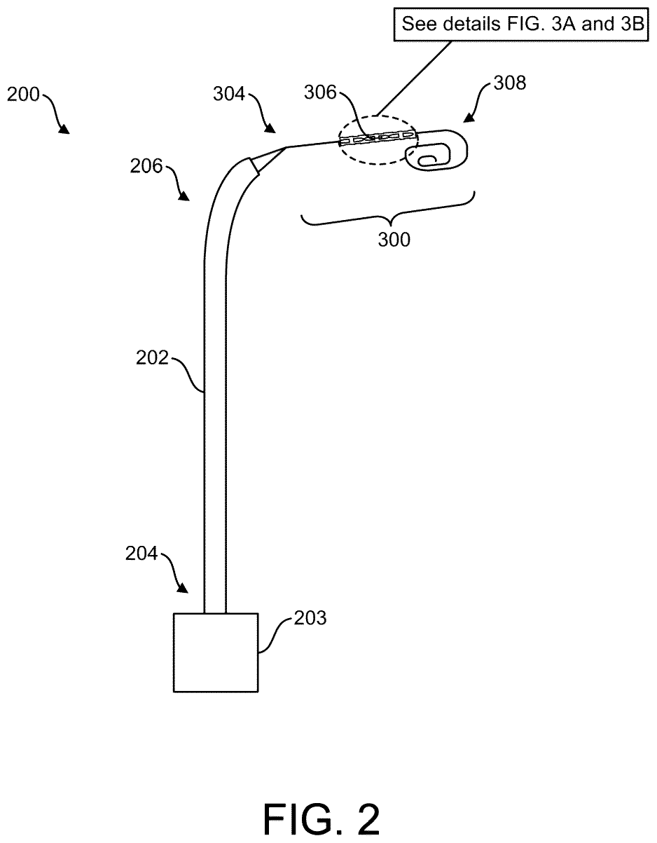

The medical device 200, which will be discussed in more detail in connection with FIG. 2, comprises an elongate guide member 202 and a guidewire 300. The elongate guide member 202 has a lumen (not shown) sized and shaped to accommodate the guidewire 300, which may be used as the component for puncturing the target tissue. In some embodiments, a puncturing tool, such as a needle or a specialized guidewire with puncturing capabilities, may be used in conjunction with the medical device 200. The puncturing tool can be inserted through the lumen of the elongate guide member 202 and advanced beyond the distal end to puncture the target tissue. The echogenic features of the guidewire 300 can help guide the positioning and advancement of the puncturing tool, ensuring accurate targeting of the desired location. After the puncture is made, the guidewire 300 can be advanced through the puncture site, allowing the elongate guide member 202 to follow and maintain access to the left side of the heart.

The guidewire 300 includes one or more echogenic features (not shown in this figure) along its distal portion, which enhance its visibility under ultrasound imaging. These echogenic features typically comprise surface discontinuities, such as indentations, protrusions, or roughened areas, that increase the reflection of ultrasound waves. By reflecting more ultrasound waves back to the ultrasound probe 108, these echogenic features make the guidewire 300 more visible within the live visual representation 104. The enhanced visibility of the guidewire 300 under the ultrasound probe 108 enables the medical team to track its position and orientation within the heart with great precision. During the transseptal puncture step, particularly after the guidewire 300 has been advanced beyond a dilator and/or sheath, it allows the operator to accurately target the preferred puncture site on the interatrial septum 350. The ability to visualize the guidewire 300 in real-time reduces the risk of inadvertent puncture of adjacent structures, such as the aorta or the posterior wall of the left atrium.

In some embodiments the operator may choose to target other regions of the heart, depending on the specific clinical scenario. For example, in patients with a history of atrial septal defect repair or a thick, fibrous septum, the operator may opt to puncture a different part of the interatrial septum or even the left atrial appendage. The echogenic features of the guidewire 300 allow for precise positioning and puncturing of these alternative sites, particularly the tracking of the guidewire up to the superior vena cava (SVC) and after the guidewire 300 has been advanced beyond a dilator and/or sheath after puncture, providing flexibility and adaptability to accommodate various patient anatomies and clinical needs.

As shown in FIG. 1, the guidewire 300 has been advanced across the interatrial septum 350 and into the left atrium 360 enabling access to the left side of the heart for further diagnostic or therapeutic interventions. The echogenic features of the guidewire 300 ensure that its position within the left atrium 360 can be clearly visualized as the ultrasound probe 108 continuously transmits ultrasound waves into the patient's chest, and the reflected sound waves are processed by the ultrasound data processor 102 to update the visual representation 104. This imaging capability allows the operating team to monitor the progress of the procedure and make any necessary adjustments to the position of the medical device 200 and allowing the operator to safely maneuver the device and perform the intended intervention.

In some embodiments, the medical device 200 may incorporate other design elements that further enhance its functionality and safety. For example, the elongate guide member may be constructed from materials that provide optimal maneuverability and stability within the heart. It may also include a mechanism for adjusting the curvature of its distal portion, allowing the operator to fine-tune the trajectory of the guidewire 300 during the puncture process.

In some embodiments, the guidewire 300 may incorporate additional markers or sensors that provide visual feedback to the operator. These may include radiopaque markers that are visible under fluoroscopy, allowing the device to be used in hybrid imaging environments. Alternatively, the guidewire 300 may be equipped with pressure sensors or other diagnostic tools that provide real-time information about the conditions within the heart.

The potential applications of the present disclosure may also extend beyond transseptal puncture procedures. The echogenic guidewire may be adapted for use in other cardiovascular interventions, such as the treatment of structural heart diseases or the delivery of cardiac therapies. Furthermore, the principles of enhanced device visibility under ultrasound imaging can be applied to other medical fields, such as neurosurgery or orthopedics, where precise device manipulation is critical.

The combination of the echogenic guidewire 300, the elongate guide member 202, and the ultrasound imaging system 100 provides a comprehensive solution for performing transseptal punctures under ultrasound guidance. By enhancing the visibility of the guidewire 300 and providing real-time visual feedback to the operator, the present disclosure aims to improve the safety, accuracy, and efficiency of transseptal puncture procedures. The detailed design and functionality of the medical device 200, including the elongate guide member 202 and the guidewire 300, will be further explored in the discussion of FIG. 2.

FIG. 2 provides a detailed illustration of the medical device 200, which enables operators to facilitate safe and accurate transseptal punctures under ultrasound guidance. The medical device 200 comprises an elongate member 202 and a guidewire 300, which work together to enable the operator to navigate the device within the patient's heart and create a controlled puncture in the interatrial septum. The elongate member 202 serves as the primary conduit for the guidewire 300, providing a stable and maneuverable pathway from the point of entry to the heart. It has a tubular structure with a proximal guide portion 204 and a distal guide portion 206. The proximal guide portion 204 remains outside the patient's body and may incorporate a handle or a hub 203 that allows the operator to control the device. In some embodiments, the handle 203 may include various controls, such as buttons, knobs, or levers, that enable the operator to adjust the position, orientation, and function of the device 200.

The medical device 200 also controls the advancement and retraction of the guidewire 300. This may be achieved through a dedicated lever or sliding mechanism on the handle 203 that is connected to the guidewire 300. By manipulating this lever, the operator can precisely control the extension and retraction of the guidewire 300 relative to the distal guide portion 206. This allows for fine-tuned positioning of the guidewire 300 during the puncture process and helps to minimize the risk of overshooting or undershooting the target site.

The elongate member 202 also includes a lumen (not shown) that runs from the proximal guide portion 204 to the distal guide portion 206. This lumen accommodates the guidewire 300 and allows for smooth and efficient advancement and retraction of the guidewire 300 during the procedure. The lumen is sized to provide a close fit around the guidewire 300, minimizing any gaps or spaces that could allow for blood leakage or air embolism.

The distal guide portion 206 of the elongate member 202 is to be inserted into the heart and positioned adjacent to the target puncture site, typically the fossa ovalis of the interatrial septum. The distal guide portion 206 may have a pre-shaped curve or a steerable tip that facilitates positioning and orientation within the heart. In some embodiments, the distal guide portion 206 may include additional features, such as radiopaque markers or electrodes, that help with visualization and navigation under fluoroscopy or other imaging modalities.

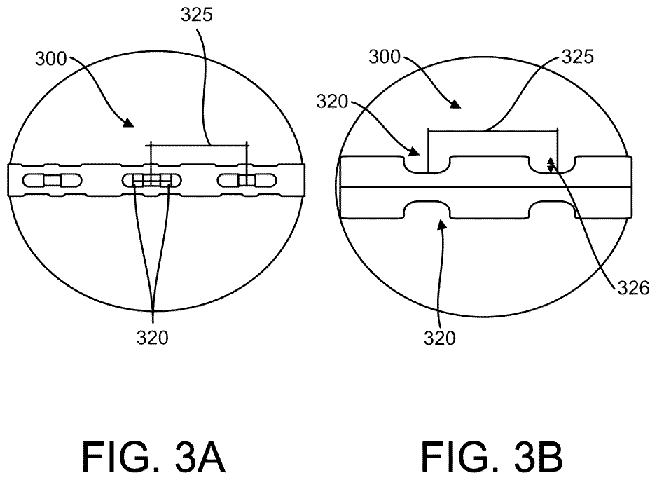

The guidewire 300 has an elongate body 306 that extends from a proximal end 304 to a distal end 308. The proximal end 304 is connected to the advancement and retraction mechanism in the handle 203 of the elongate member 202, allowing for control of the guidewire 300 position. The distal end 308 of the guidewire 300 may comprise one or more echogenic features 320 (see FIGS. 3A and 3B). The specific configuration of the distal end 308 can vary depending on the intended application and the puncture technique being used (the terms “echogenic features” and “echogenic markers” are used interchangeably through the description to refer to the surface discontinuities on the guidewire 300).

In some embodiments, the shape of the distal end 308 may be modifiable by the operator during the procedure. This can be achieved using a control mechanism, such as a lever or a knob, located on the handle 203. The control mechanism is typically connected to a cord or a wire that extends within the guidewire 300 and is attached to the distal end 308. By manipulating the control mechanism, the operator can increase or decrease the tension on the cord, thereby altering the shape of the distal end 308. This allows the operator to fine-tune the position of the distal end 308 within the heart, ensuring that it is directed away from sensitive structures and minimizing the risk of complications.

In some embodiments, the distal end 308 of the guidewire 300 may have a sharp, needle-like tip that is designed for mechanical puncture of the septum. This type of tip is often used in conjunction with a conventional transseptal needle, where the guidewire 300 is advanced forcefully through the septum to create the puncture. The sharp tip concentrates the force at a small point, allowing the guidewire 300 to penetrate the tough septal tissue.

In some embodiments, the distal end 308 of the guidewire 300 may comprise one or more electrodes configured for radiofrequency (RF) or other energy-based puncture techniques. The one or more electrodes are connected to an external energy source, such as an RF generator. When activated, the electrodes deliver high-frequency electrical energy to the tissue, causing localized heating and tissue vaporization. This creates a controlled passage through the septum, allowing the guidewire 300 to advance into the left atrium. The electrode configuration may have various designs, such as a monopolar or bipolar, and may include features such as temperature sensors or impedance monitoring to ensure safe and effective energy delivery.

Regardless of the specific configuration of the distal end 308, the distal end 308 of the guidewire 300 includes one or more echogenic features 320 that enhance its visibility under ultrasound imaging. These features, which are described in more detail in FIGS. 3A and 3B, may take the form of surface discontinuities, such as grooves, indentations, or protrusions, that increase the scattering and reflection of ultrasound waves. By reflecting more ultrasound waves back to the imaging probe, the echogenic features 320 make the guidewire 300 stand out within the visual representation of the heart, allowing the operator to track its position and orientation.

In addition to the echogenic features 320, the guidewire 300 may include other elements that enhance its performance and safety. In some embodiments, a pressure sensor or a temperature sensor may be incorporated near the distal end 308, allowing the operator to monitor the conditions within the heart and detect any signs of complications. In some embodiments, there may be a steering mechanism, such as a pull wire or a shape memory alloy, that allows the operator to adjust the curvature of the distal end 308 during the procedure.

The medical device 200 may also include additional components that facilitate the transseptal puncture process, such as a dilator or a sheath (not shown). The dilator may be designed to work in conjunction with the guidewire 300 to facilitate the transseptal puncture process. The dilator may include a central lumen that is sized to accommodate the guidewire 300, allowing the two components to be advanced together as a single unit.

D During the procedure, the guidewire 300 may be advanced through the dilator until its distal end 308 extends beyond the tip of the dilator. The echogenic features 320 on the distal end 308 of the guidewire 300 enable precise positioning of the guidewire tip at the target puncture site, typically the fossa ovalis of the interatrial septum. Once the guidewire 300 is in position, the dilator may be advanced over the guidewire 300 until its tip contacts the septum.

The dilator would provide additional support and stability to the guidewire 300, helping to maintain its position at the target site. Further, the dilator may have a tapered profile that helps to gradually dilate the puncture site as it is advanced, reducing the force required to penetrate the septum and minimizing the risk of tissue damage. Once the dilator has been advanced through the septum and into the left atrium, a sheath may be advanced over the dilator to maintain access to the left atrium. The dilator and guidewire 300 can then be removed, leaving the sheath in place as a conduit for the introduction of other devices, such as catheters, electrodes, or implants, depending on the specific clinical application.

The sheath and dilator may be designed with additional features that enhance their performance and safety during the transseptal puncture procedure. They may include surface discontinuities such as indentations, protrusions, or radiopaque markers or other visualization aids that facilitate their positioning and advancement under fluoroscopic or echocardiographic guidance. They may also incorporate materials or coatings that enhance their lubricity, biocompatibility, or resistance to kinking or fracture.

In some embodiments, the dilator may be designed to work specifically with the guidewire 300 to enhance the precision and safety of the transseptal puncture. For example, the dilator may include echogenic features such as surface discontinuities that aligns with the echogenic features 320 on the guidewire 300, providing additional confirmation of the guidewire position at the target site. Alternatively, the dilator may incorporate a pressure sensor or other monitoring device that helps to detect the moment of puncture and prevent overshoot or excessive force application.

In some embodiments, the medical device 200 may be used in conjunction with other imaging modalities, such as fluoroscopy or intracardiac echocardiography (ICE), to provide additional guidance and visualization during the procedure. The elongate member 202 and the guidewire 300 may include radiopaque markers or other features that are visible under these imaging modalities, allowing for seamless integration with existing clinical workflows.

While the present disclosure has been described with specific reference to a guidewire 300, the principles and techniques disclosed herein can be applied to a wide range of medical devices that feature a round mandrel or elongate cylindrical structure. The echogenic features 320, created through the various methods such as laser etching, chemical etching, multi-way crimping, multi-way welding, material electro-spark deposition, grinding, grooving, knurling, threading, selective grit blasting, sink EDM, additive molding, or laser cutting, can be incorporated into any round mandrel to enhance its visibility under ultrasound imaging. This includes, but is not limited to, catheters, dilators, sheaths, and other elongate medical devices. The incorporation of echogenic features onto these devices can improve their visualization and navigation during ultrasound-guided procedures, enhancing the safety, accuracy, and efficiency of various interventional applications.

FIG. 3A and FIG. 3B provide a detailed view of the guidewire 300, focusing on the surface discontinuities or echogenic markers 320 that are incorporated along the wire. These illustrations offer a closer look at the guidewire 300 and its surface discontinuities or echogenic markers 320, which aims to enhance the visibility and detectability of the guidewire 300 under ultrasound imaging by increasing the scattering and reflection of ultrasound waves back towards the imaging probe. Each echogenic marker may be created using two closely spaced points of surface deformation. Although two physical indentations are created (as shown), they appear as a single marker under ultrasound imaging due to their proximity. FIG. 3A presents a top view of the echogenic markers 320 on the guidewire 300, while FIG. 3B offers a side view, revealing the depth 326 of the echogenic feature and the distance 325 between the two points of deformation. It is important to note that the distance 325 shown is center-to-center between the two sets of markers.

The echogenic markers 320 can be created using a variety of methods, each designed to modify the smooth, uniform surface of the guidewire 300 in a controlled and predictable manner. These methods include mechanical deformation techniques such as laser etching, chemical etching, multi-way crimping, multi-way welding, material electro-spark deposition, grinding, grooving, knurling, threading, selective grit blasting, sink EDM, additive molding, laser cutting, or the application of echogenic coatings or materials. The choice of method depends on factors such as the desired pattern, visibility, and spacing of the markers, as well as the material properties of the guidewire 300.

One method for creating the echogenic markers 320 is a multi-step mechanical crimping process. In this approach, the length of the guidewire 300 or a portion of the guidewire 300, such as the distal end 308 of the guidewire 300, is subjected to a series of controlled mechanical deformations using a specialized crimping machine equipped with dies or indenters. These tools apply precise, localized pressure to the guidewire 300, causing the material to deform plastically and create a specific pattern of indentations or surface discontinuities such as protrusions, texturing, or any other alterations on the surface.

The crimping process can be optimized to achieve the desired echogenic performance while maintaining the mechanical integrity and functionality of the guidewire 300. By adjusting parameters such as the configuration of the dies, the force applied, and the number of crimping steps, various patterns, shapes, and depths of echogenic markers 320 can be created. In one embodiment, the crimping process may yield a series of evenly spaced indentations or surface discontinuities along the length of the guidewire 300 or a portion of the guidewire 300, such as the distal end 308 of the guidewire 300, with each indentation having a controlled depth 326 and a specific shape or profile.

The distance 325 between the individual echogenic markers 320 can have influences on the guidewire's visibility under ultrasound imaging. Optimal distance enhances the guidewire's visibility and allows for better tracking of its movements during procedures. When the distance between markers is sufficiently large, it creates a clear contrast between the echogenic markers and the unmarked wire segments. Conversely, if the distance between the markers is too small, the makers may blend together, potentially obscuring the guidewire's motion and visualization. Further more, the distance 325 should be large enough to ensure that the guidewire 300 appears as a continuous, highly echogenic structure, but small enough to prevent excessive deformation or weakening of the guidewire material. In some embodiments, the distance 325 between the markers is approximately 2-6 mm, which provides a balance between echogenicity and mechanical integrity.

Similarly, the depth 326 of each echogenic marker 320 influences the guidewire's echogenicity. Deeper markers generally result in greater scattering of ultrasound waves, leading to higher echogenicity. However, excessively deep markers may compromise the mechanical strength or flexibility of the guidewire 300. In some embodiment, the depth 326 of the echogenic markers 320 ranges from approximately. 0005 inches to .002 inches ensuring a balance between echogenicity and maintaining the guidewire's performance. In certain embodiments, the echogenic markers 320 has a target depth of approximately .00125 inches with a permissible variation of +/−. 00075 inches representing an enhanced ultrasound visibility while preserving the guidewire's structural integrity.

The shape or profile of the individual echogenic markers 320 can also be optimized to enhance the guidewire's visibility under ultrasound imaging. The crimping process may produce markers with various cross-sectional shapes, such as trapezoidal, triangular, or rounded profiles. In some embodiments, the markers are created as a series of circular or oval-shaped indentations that are evenly distributed around the circumference of the guidewire 300, ensuring visibility regardless of the guidewire's rotational orientation within the body.

In some embodiments, creating at least two indentations or surface discontinuities that are spaced 90° apart from each other around the circumference of the length of the guidewire 300 or a portion of the guidewire 300, such as the distal end 308 of the guidewire 300, may maximize echogenicity. This four-way indent crimp pattern ensures that at least one echogenic marker 320 is always perpendicular to the incident ultrasound beam, maximizing the scattering and reflection of ultrasound waves in the direction of the imaging probe and enhancing the visibility and detectability of the guidewire 300.

In addition to the mechanical crimping process, other methods such as laser etching or cutting, chemical etching, and abrasive techniques like grit blasting or grinding can be employed to create the echogenic markers 320. These methods can be used alone or in combination to achieve the desired surface modification and echogenic performance.

The choice of guidewire material can also influence the effectiveness of the echogenic markers 320. In some embodiments, the guidewire 300 is made of stainless steel, which provides a balance of strength, flexibility, and echogenicity. Other materials, such as nitinol or titanium, may be used to achieve specific mechanical properties or to enhance the guidewire's compatibility with certain imaging modalities.

The incorporation of echogenic markers 320 onto the guidewire 300 offers improved visibility and detectability under ultrasound imaging. By controlling the spacing 325, depth 326, shape, and pattern of the individual markers, as well as the material of the guidewire 300, it is possible to optimize the echogenic performance for a wide range of clinical applications and imaging scenarios.

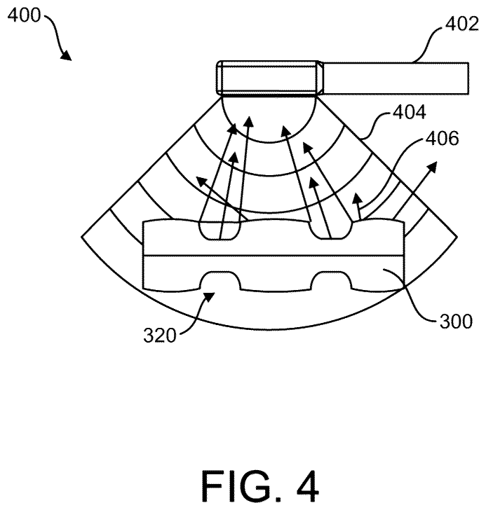

FIG. 4 provides a schematic representation 400 of the interaction between an ultrasound imaging system and the guidewire 300 with echogenic markers 320. This illustration details the enhanced visibility and detectability of the guidewire 300 achieved through the incorporation of surface discontinuities or echogenic markers 320.

The ultrasound imaging system may typically consist of an ultrasound device or probe 402 that emits high-frequency sound waves, known as ultrasound waves 404, into the patient's body. When these ultrasound waves 404 encounter the guidewire 300, they interact with its surface, and the presence of the echogenic markers 320 influences the way the waves are reflected and scattered.

In the case of a guidewire with a smooth surface, the ultrasound waves 404 emitted by the ultrasound device or probe 402 would interact with the uniform surface in a predictable, specular manner. The majority of the ultrasound energy would be reflected at an angle equal to the angle of incidence, resulting in a relatively weak signal returning to the ultrasound probe 402. This weak signal can make it challenging to visualize and track the smooth guidewire, particularly in the presence of surrounding tissue or anatomical structures.

In contrast, the guidewire 300 with echogenic markers 320 presents a non-uniform surface that alters the way ultrasound waves 404 are reflected and scattered. The surface discontinuities created by the echogenic markers 320, whether in the form of indentations, raised edges, protrusions, or a combination, cause the ultrasound waves to be reflected and deflected in various directions, as indicated by the reflection arrows 406 in FIG. 4. This increased scattering of ultrasound energy results in a stronger signal returning to the ultrasound probe 402, enhancing the overall echogenicity and visibility of the guidewire 300.

The size, shape, and spacing of the echogenic markers 320 may help determining the extent of ultrasound wave scattering and, consequently, the level of echogenicity enhancement. Larger indentations or protrusions tend to scatter ultrasound waves more effectively than smaller ones, as they present a greater surface area for interaction. However, the depth and size of the echogenic markers 320 must be carefully balanced to ensure that the structural integrity and mechanical properties of the guidewire 300 are not compromised.

The shape of the echogenic markers 320 also influences the scattering pattern of the ultrasound waves. Irregular shapes, such as those created by mechanical deformation techniques like crimping or indenting, can provide a more complex scattering profile compared to regular shapes, such as those produced by laser etching or chemical etching. The choice of shape and pattern of the echogenic markers 320 can be optimized based on the specific requirements of the interventional procedure and the imaging modality being used.

In addition to the size and shape of the echogenic markers 320, the spacing between the echogenic markers 320 also affects the overall echogenicity of the guidewire 300. A higher density of echogenic markers 320 along the length of the guidewire 300 can result in a more consistent and continuous enhancement of echogenicity. However, the spacing must be carefully considered to avoid excessive deformation or weakening of the guidewire material.

In some embodiments, coatings and surface treatments can also be applied to the guidewire 300 to further enhance its echogenicity. For example, applying a coating containing echogenic particles, such as microbubbles or metallic nanoparticles, can increase the scattering of ultrasound waves and improve the visibility of the guidewire 300. These coatings can be used in conjunction with the echogenic markers 320 to provide an additional level of echogenicity enhancement.

It is important to note that the optimal configuration of echogenic markers 320, including their size, shape, spacing, and any additional coatings or surface treatments, may vary depending on the specific interventional application and the ultrasound imaging system being used. Factors such as the frequency of the ultrasound waves, the depth of the target anatomical structure, and the presence of surrounding tissues must be considered when creating the echogenic markers 320 to ensure the best possible visualization and tracking of the guidewire 300.

Various imaging modalities can benefit from the enhanced echogenicity provided by the echogenic markers 320 on the guidewire 300. In addition to conventional 2D ultrasound imaging, other advanced techniques such as intracardiac echocardiography (ICE), transesophageal echocardiography (TEE), and 3D ultrasound imaging can leverage the improved visibility of the guidewire 300 to facilitate accurate navigation, positioning, and monitoring during complex interventional procedures.

The incorporation of echogenic markers 320 onto the guidewire 300 helps transseptal puncture procedures, where precise positioning and crossing of the interatrial septum are important for successful outcomes. The enhanced visibility of the guidewire 300 under ultrasound imaging reduces the reliance on fluoroscopic guidance, minimizing radiation exposure to both patients and medical personnel.

In some embodiments, the improved echogenicity of the guidewire 300 can be beneficial in peripheral vascular interventions, where accurate navigation and positioning of the guidewire are important for treatment success. The echogenic markers 320 enable better visualization of the guidewire 300 in relation to the surrounding anatomical structures, facilitating more precise and efficient interventions.

As mentioned in the discussion of FIG. 3A and FIG. 3B, various methods can be employed to create the echogenic markers 320 on the guidewire 300, including mechanical deformation techniques (e.g., crimping, indenting, or scoring), laser etching, chemical etching, and additive methods like coating or plating. Each of these methods offers different terms of precision, controllability, and the ability to create specific patterns and shapes that optimize the scattering of ultrasound waves.

By leveraging the scattering properties of surface discontinuities improves the visibility and detectability of the guidewire 300 under various ultrasound imaging modalities, leading to safer, efficient, and effective interventional procedures.

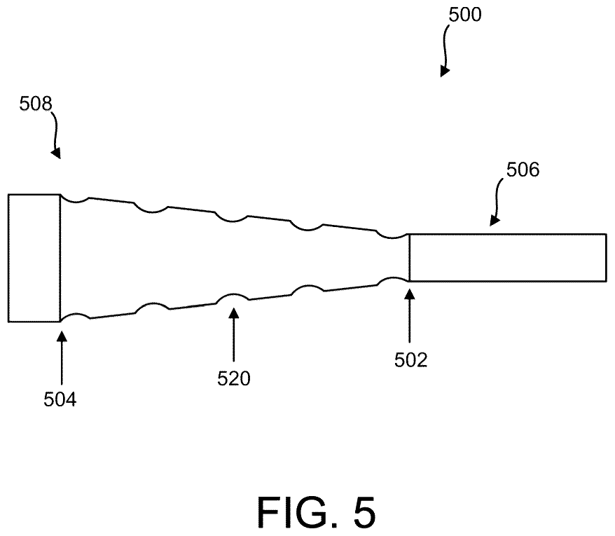

FIG. 5 illustrates a guidewire 500 that incorporates echogenic markers 520 along its length while also featuring distinct regions of varying stiffness combining the enhanced visibility provided by the echogenic markers 520 with the functional benefits of a guidewire that has a specific stiffness profile, making it suitable for a wide range of interventional procedures.

The guidewire 500 comprises a proximal end 508 and a distal end 506, with the stiffness of the guidewire varying along its length. As shown in FIG. 5, the guidewire 500 has a region of minimum stiffness 502 located at the distal end 506 and a region of maximum stiffness 504 located at the proximal end 508. This stiffness profile, known as a “tapered” configuration, allows for a gradual increase in stiffness from the distal end to the proximal end.

The tapered configuration offers a more flexible distal end 506, represented by the region of minimum stiffness 502, enabling the guidewire to navigate through blood vessels and anatomical structures more easily, and reducing the risk of tissue damage or perforation. The guidewire 500 can conform to the shape of the vasculature without exerting excessive force on the vessel walls. Conversely, the stiffer proximal end 508, represented by the region of maximum stiffness 504, provides the necessary pushability and torque control to advance the guidewire 500 through the vasculature and cross lesions or stenoses. The increased stiffness at the proximal end also enhances the stability of the guidewire 500 during the delivery and deployment of interventional devices, such as stents or balloon catheters.

The echogenic markers 520 incorporated along the length of the guidewire 500 serve to enhance its visibility and detectability under ultrasound imaging. These markers create surface discontinuities that increase the scattering of ultrasound waves, resulting in a stronger signal returning to the ultrasound probe. The enhanced echogenicity of the guidewire 500 facilitates accurate navigation, positioning, and tracking during interventional procedures.

In some embodiments, the echogenic markers 520 may be positioned along the guidewire 500, taking into account the varying stiffness profile. The markers 520 may be more widely spaced in the region of minimum stiffness 502 to ensure adequate visibility of the flexible distal end, which is more likely to undergo significant bending and deformation during navigation. Conversely, the echogenic markers 520 may be more closely spaced in the region of maximum stiffness 504, as this portion of the guidewire 500 is less likely to experience significant flexing.

The echogenic markers 520 are distributed around the circumference of the guidewire 500, ensuring visibility from all angles under ultrasound imaging. By having markers 520 on all sides of the guidewire 500, rotated 90° apart from each other, ensures that at least one set of markers is always favorably oriented towards an imaging probe and the visibility is maintained regardless of the guidewire's rotational position at different orientations within the vasculature.

The specific pattern, spacing, and configuration of the echogenic markers 520 can be optimized based on the intended application of the guidewire 500. For instance, in neurovascular interventions, where the guidewire must navigate through small, tortuous blood vessels in the brain, a higher density of echogenic markers 520 may be desirable to ensure precise tracking and positioning. In contrast, for peripheral vascular interventions involving larger blood vessels, a lower density of echogenic markers 520 may be sufficient to provide adequate visibility while minimizing the impact on the guidewire's mechanical properties.

In addition to the tapered configuration shown in FIG. 5, other stiffness profiles can be employed in conjunction with the echogenic markers 520 to suit specific interventional needs. In some embodiments, a guidewire with a more abrupt transition between the stiff proximal end and the flexible distal end, such as a “stepped” configuration, may be preferred in certain applications where rapid changes in stiffness are required. In some embodiments, a guidewire with a more gradual, continuous change in stiffness along its length, referred to as a “progressive” configuration, may be advantageous in situations where a smoother transition between flexibility and pushability is desired.

The incorporation of echogenic markers 520 onto a guidewire 500 with varying stiffness regions offers benefits in terms of both visibility and performance. By combining the enhanced echogenicity provided by the markers with the functional advantages of a tapered or otherwise optimized stiffness profile, interventional procedures can be performed with greater precision, safety, and efficiency.

The specific configuration of the guidewire 500, including the stiffness profile, the arrangement of the echogenic markers 520, and their circumferential distribution, may vary depending on the intended application, the target anatomical location, and the preferences of the interventional operator. Factors such as the size and tortuosity of the blood vessels, the presence of lesions or chronic total occlusions, and the type of interventional devices being used must be considered when selecting the most appropriate guidewire design for treatment delivery.

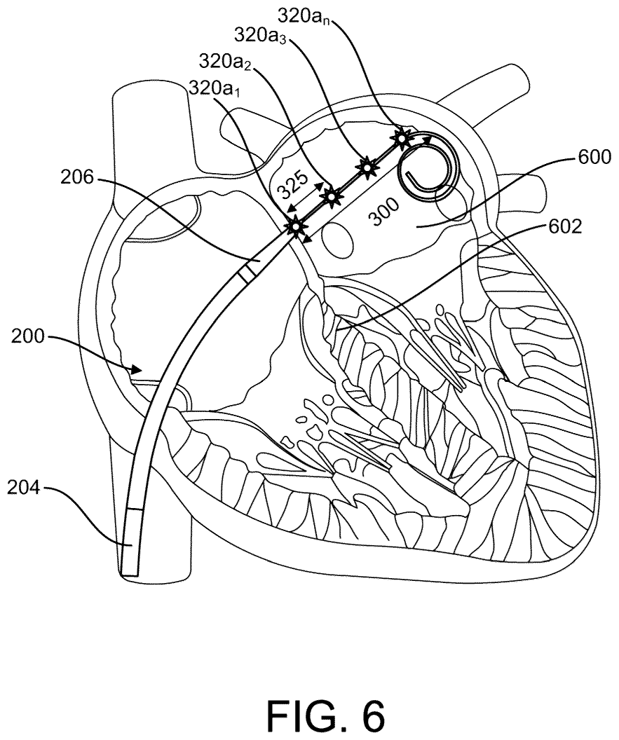

FIG. 6 illustrates the medical device 200 in use within the heart, demonstrating the versatility and utility of the echogenic markers 320 on the guidewire 300. As shown in FIG. 6, the guidewire 300 is passing through the septum 602 and into the left atrium 600. The medical device 200 comprises an elongate member 202 with a proximal guide portion 204 and a distal guide portion 206. The elongate guide member 202 has a lumen (not shown) sized and shaped to accommodate the guidewire 300. The guidewire 300, which features the echogenic markers 320, extends beyond the distal guide portion 206, penetrating the septum 602 and entering the left atrium 600.

As shown in FIG. 6, the echogenic markers 320 on the guidewire 300 serves as a measurement guide for estimating the size of various anatomical structures within the heart. The use of echogenic markers 320 as a measurement guide can be used in procedures where accurate sizing is needed, such as left atrial appendage closure or pulmonary vein isolation. The guide wire 300 may contain a plurality of echogenic markers 320 such as 320a₁, 320a₂, 320a₃, and 320aₙ. The value of “n” can be any positive integer, allowing for flexibility in the design and adaptation of the guidewire 300 to accommodate different numbers of echogenic markers 320. These echogenic markers 320a₁ through 320aₙ are evenly spaced apart by a distance 325. By knowing the spacing 325 between the markers, medical operators can quickly and accurately estimate the dimensions of structures such as the left atrial appendage or the pulmonary veins. By aligning the guidewire 300 with the structure of interest and counting the number of markers 320 that span the structure, physicians can obtain a rapid assessment of its size without the need for additional imaging or measurement tools.

Moreover, the echogenic markers 320 can also be used to determine the insertion depth of accessory devices, such as dilators, sheaths, or catheters, that are advanced over the guidewire 300. As these devices are advanced, they progressively cover the echogenic markers 320 on the guidewire 300. By monitoring the number of markers 320 that remain visible under ultrasound imaging, physicians can precisely control the insertion depth of the accessory devices.

This feature may be useful in transseptal procedures, where the accurate positioning of the dilator and sheath is needed for safe and effective puncture of the septum 602. By using the echogenic markers 320 as a reference, medical operators can ensure that the dilator and sheath are advanced to the optimal position before performing the transseptal puncture.

In addition to the measurement and positioning capabilities, the echogenic markers 320 on the guidewire 300 also enhance its overall visibility under ultrasound imaging, as described in the previous figures. The improved visualization of the guidewire 300 within the heart facilitates more precise navigation, reduces the risk of complications, and increases the overall safety and efficiency of the procedure.

To further enhance the functionality and user experience of the medical device 200, various embodiments may be incorporated such as a handle with a control mechanism or lever. For example, a pull wire or other actuation mechanism could be integrated into the handle, allowing the physician to adjust the curvature or shape of the distal guide portion 206. This feature would enable better maneuverability and positioning of the guidewire 300 within the complex anatomy of the heart.

In some embodiments, different patterns, spacings, or configurations of the echogenic markers 320 to suit specific clinical applications or physician preferences. For instance, the spacing 325 between the markers could be adjusted to correspond with commonly encountered anatomical dimensions, or the markers could be arranged in different patterns to enhance visibility from multiple angles.

The echogenic markers 320 on the guidewire 300 may serve more than one function such as a measurement guide and positioning aid in transseptal procedures and left atrial interventions. By leveraging the evenly spaced markers 320a₁, 320a₂, 320a₃, and 320aₙ, medical operators can quickly estimate the size of anatomical structures and precisely control the insertion depth of accessory devices. The improved visualization and functionality of the medical device 200 contribute to safer, more efficient, and more effective procedures within the complex anatomy of the heart.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device for use in conjunction with an elongate guide member defining a lumen and having a proximal guide portion and a distal guide portion, the device comprising:
a guidewire configured to be disposed within the lumen, the guidewire having an elongate body extending from a proximal end to a distal portion having a distal end,
wherein the distal portion of the elongate body includes at least one echogenic feature, and
wherein the at least one echogenic feature comprises a discontinuity configured to reflect ultrasonic waves so as to enhance visibility of the inner wire under ultrasound imaging.

2. The medical device of claim 1, wherein the at least one echogenic feature comprises a plurality of echogenic markers formed by mechanically deforming the surface of the distal portion of the elongate body to create a plurality of discontinuities.

3. The medical device of claim 2, wherein mechanically deforming the surface of the distal portion of the elongate body comprises creating a pattern of deformed material on the surface by forming indentations.

4. The medical device of claim 2, wherein mechanically deforming the surface of the distal portion of the elongate body comprises at least one of laser etching, chemical etching, multi-way crimping, multi-way welding, material electro-spark deposition, grinding, grooving, knurling, threading, selective grit blasting, sink EDM, additive molding, or laser cutting.

5. The medical device of claim 3, wherein the indentations are configured to scatter ultrasound waves by presenting a non-uniform surface that reflects ultrasound waves in various directions.

6. The medical device of claim 3, wherein the mechanical deforming creates at least two indentations in line, spaced 90° apart from each other around a circumference of the distal portion of the elongate body.

7. The medical device of claim 2, wherein the plurality of echogenic markers are evenly spaced along a length of the distal portion of the elongate body.

8. The medical device of claim 7, wherein the evenly spaced echogenic markers are configured to provide a visual reference for measuring anatomical features or an insertion depth of the guidewire under ultrasound imaging.

9. The medical device of claim 2, wherein the plurality of the echogenic markers are positioned to indicate different stiffness regions of the guidewire.

10. The medical device of claim 2, wherein a spacing between the plurality of the echogenic markers is selected to enable visualization of a relative motion of the guidewire under ultrasound imaging.

11. The medical device of claim 1, wherein the guidewire is configured to be advanced out of the distal guide portion of the elongate guide member.

12. The medical device of claim 1, wherein the elongate guide member comprises a dilator having a tapered distal end configured to facilitate advancement through the septum.

13. The medical device of claim 12, wherein the dilator further comprises at least one echogenic feature disposed on a surface of the dilator to enhance visibility of the dilator under ultrasound imaging.

14. The medical device of claim 1, wherein the elongate guide member comprises a sheath having a distal end, wherein the sheath comprises at least one echogenic feature disposed on a surface of the sheath.

15. The medical device of claim 1, wherein the distal end of the guidewire comprises at least one electrode configured to deliver radiofrequency energy for tissue vaporization during transseptal puncture.

16. An ultrasound imaging system comprising:
an ultrasound transducer configured to emit and receive ultrasound signals; and
a medical device for use in conjunction with an elongate guide member defining a lumen and having a proximal guide portion and a distal guide portion to cross a septum of a patient's heart, the device comprising:
a guidewire configured to be disposed within the lumen, the guidewire having an elongate body extending from a proximal end to a distal portion having a distal end,
wherein the distal portion of the elongate body includes at least one echogenic feature, and
wherein the at least one echogenic feature comprises a discontinuity configured to reflect ultrasonic waves so as to enhance visibility of the inner wire under ultrasound imaging.

17. The ultrasound imaging system of claim 16, wherein the at least one echogenic feature comprises a plurality of echogenic markers formed by mechanically deforming a surface of the distal portion of the elongate body to create the plurality of discontinuities.

18. The ultrasound imaging system of claim 17, wherein mechanically deforming the surface of the distal portion of the elongate body comprises at least one of laser etching, chemical etching, multi-way crimping, multi-way welding, material electro-spark deposition, grinding, grooving, knurling, threading, selective grit blasting, sink EDM, additive molding, or laser cutting.

19. The ultrasound imaging system of claim 17, wherein mechanically deforming the surface of the distal portion of the elongate body comprises creating a pattern of deformed material on the surface by forming indentations.

20. A method for manufacturing a medical device with enhanced visibility under ultrasound imaging, the method comprising:
providing an elongate guide member defining a lumen and having a proximal guide portion and a distal guide portion;
providing a guidewire having an elongate body extending from a proximal end to a distal portion having a distal end; and
forming at least one echogenic feature on the distal portion of the elongate body by mechanically deforming a surface of the distal portion to create a plurality of discontinuities that increase ultrasound wave reflection or beam scattering, thereby enhancing visibility of the guidewire under ultrasound imaging.
